(19) United States
(12) Patent Application Publication
(10) Pub. No.: US 2016/0209406 A1
(43) Pub. Date: Jul. 21, 2016

(54) APPARATUS FOR DETECTING AN ANALYTE USING A FLUID HAVING MAGNETIC PARTICLES WITH A BINDING LIGAND FOR BINDING THE ANALYTE

(71) Applicant: ANMAT TECHNOLOGY LIMITED, Oldham, Lancashire (GB)

(72) Inventors: Harmesh Singh Aojula, Oldham (GB); David John Clarke, Sandbach (GB)

(21) Appl. No.: 14/914,514

(22) PCT Filed: Aug. 15, 2014
(86) PCT No.: PCT/GB2014/000316

§ 371 Date: Feb. 25, 2016

(30) Foreign Application Priority Data
Aug. 28, 2013 (GB) 1315327.5

(51) Int. Cl. G01N 33/543; G01N 21/64; G01R 33/12

(52) U.S. Cl. CPC G01N 33/54326; G01N 33/54373; G01R 33/1276; G01N 21/6428; G01N 2201/062; G01N 2021/6439

(57) ABSTRACT

Apparatus for detecting an analyte using a fluid having magnetic particles with a binding ligand for binding the analyte, which apparatus comprises: (i) a compartment (1); (ii) an inlet port (6) for allowing the analyte in the fluid to enter the compartment (1); (iii) a wall in the compartment (1) with at least of the part of the wall being optically transparent; and (v) a first magnet which is positioned outside of the compartment (1), and which is for transporting and positioning the magnetic particles (2) with the binding agent and the analyte to an imaging plane where the particles are able to be illuminated for the detection of the analyte. A method for detecting the analyte is also described.

This invention relates to apparatus and a method for detecting an analyte. More especially, this invention relates to apparatus and a method for detecting an analyte using a fluid having magnetic particles with a binding ligand for binding the analyte.

Particles are transported in different types of fields for the purposes of separation and analysis, such as by electrophoresis, dielectrophoresis, ultrasound and magnetophoresis. Magnetic particles (e.g super-paramagnetic particles) are most widely used to separate analyte from samples [Beveridge J S, Stephens J R & Williams M E 2011) The Use of Magnetic Nanoparticles in Analytical Chemistry. Annual Review of Analytical Chemistry 4:251-273]. Typically, analytes are collected using binding agents such as antibodies immobilised on the magnetic particles, when the magnetic particles are collected proximal to a magnet, the sample discarded and the magnetic particles washed by repeated addition and removal of media to remove residual sample. The magnetic particles may then be subjected to a variety of analytical and assay methods, such as microbiological, immunoassays, polymerase chain reaction and probes for assay of DNA and RNA.

Particle luminescence is widely used in assays, including by using luminescent particles as labels or by binding fluorescent labels to analytes bound to particles [Kim J S & Ligler F S (2010) Utilization of microparticles in next-generation assays for microflow cytometers. Anal Bioanal Chem 3986: 2373-2382], using a variety of optical apparatus requiring arrangements of multiple lenses and filters [Lacowicz J R (2006) Principles of fluorescence spectroscopy: 2. instrumentation for fluorescence spectroscopy, Springer].

Particle separation, luminescent assay and image analysis methods are routinely performed in laboratories using extensive manual manipulation within and between each step of separation, assay and imaging or particle analysis. Robotic mechanisms are used to integrate and automate such laboratory methods.

The above laboratory and automated methods and associated apparatus have proved difficult to develop for assays in the field or in the factory or at the point of care, primarily for reasons of their relatively high capital cost and complexity, requiring a high degree of technical expertise. Instead, a variety of alternative methods have developed, such as consumable ‘dipistick’ tests, biosensor and microfluidic methods [Alphonsus H, Ng C, Uddayasankar U & Wheeler A R 2010) Immunoassays in microfluidic systems. Anal Bioanal Chem 397:991-1007], which require lower levels of technical expertise and lower cost apparatus, more suited to application outside the laboratory. In such devices, similarly to automated laboratory methods, fluid samples containing analytes flow within the device, with a stationary solid phase involved in separation and detection of analyte, including where magnetic particles are used [Wang Z, Wang X, Liu S, Yin J & Wang H (2010) Fluorescently Imaged Particle Counting Immunoassay for Sensitive Detection of DNA Modifications. Anal. Chem. 82: 9901-9908]. However, such field and point of care methods fail or struggle to achieve the performance of laboratory methods, leaving many assays in the laboratory or as screening devices, with a requirement for validation in the laboratory, to produce information that is relied upon to take action at the point of care or in the field or factory.

### DESCRIPTION OF THE INVENTION

It is an aim of the present invention to obviate or reduce the above mentioned problems.

Accordingly, in one non-limiting embodiment of the present invention there is provided apparatus for detecting an analyte using a fluid having magnetic particles with a binding ligand for binding the analyte, which apparatus comprises:
(i) a compartment;
(ii) an inlet port for allowing the analyte in the fluid to enter the compartment;
(iii) a wall in the compartment, with at least a part of the wall-being optically transparent; and
(iv) a first magnet which is positioned outside of the compartment, and which is for transporting and positioning the magnetic particles with the binding agent and the analyte to an imaging plane where the particles are able to be illuminated for the detection of the analyte.

The apparatus of the present invention is firstly advantageous in that it concentrates the analyte in the imaging plane of the detection. The apparatus is secondly advantageous in that it is able to move the analyte out of the background turbidity/interference of the fluid. The apparatus is thirdly advantageous in that the particles are more visible and therefore able to be counted. The counting gives more sensitivity than is otherwise often possible, and thus it becomes possible to measure things that would previously not able to be measured. The apparatus is fourthly advantageous in that the ability to measure with the counting enables measurements to be made outside of a laboratory and in a more simple manner than hitherto. Thus, for example, micro-organisms may be measured. The present invention may be regarded as integrating separation and assay of analytes into a single low cost apparatus and method, with the performance of automated laboratory methods, and Which is suitable for use with minimal technical skill.

The apparatus of the invention may include a reflective surface which in use is in the compartment and opposite the part of the wall that it optically transparent, the reflective surface allowing the particles to be illuminated and thereby become visible above background illumination.

The apparatus may include a second magnet which provides the reflective surface. The second magnet may protrude into the fluid and thereby reduce the measurement path length.

Alternatively, the apparatus may be one in which the reflective surface is part of a wall of the compartment: In this case, the reflective surface may be, for example, an air:water reflective surface, a mirrored reflective surface, a wave guide, or other type of reflective surface.

The first magnet may be a stationary magnet. Alternatively, the first magnet may be a moveable magnet.

The apparatus may include imaging optics for the detection of the analyte. Various types of imaging optics may be employed. Thus, for example, the imaging optics may include a telecommunications device. The telecommunications device may be various types of cameras including mobile telephones with a camera facility. The use of such imaging optics enables the apparatus of the present invention readily to be used by persons outside of a laboratory, for example at home. Alternatively, the imaging optics may be of a complex bespoke nature for use of the apparatus of the present invention by professional persons skilled in the art of detecting analytes.

The apparatus may be one in which the wall of the compartment having the part which is optically transparent may be such as to form optical connection means to imaging means, for example a dedicated camera or a mobile telephone with an inbuilt camera.

The first magnet may be a moveable magnet which moves relative to the compartment. Alternatively, the first magnet may be a fixed magnet which is fixed with respect to a moveable compartment.

The apparatus may include illumination means for illuminating the particles for the detection of the analyte.

The illumination means may be a polarising means. Other types of polarising means may be employed such for example as red, blue and green light-emitting diodes.

The apparatus may be one in which the compartment is in a sample holder. In this case, the compartment may be within the sample holder or in a side compartment of the sample holder.

The apparatus may include introducing means for introducing the fluid.

The introducing means may be a dropper of a type which concentrates the fluid, or it may be an arrangement which keeps the fluid stationary in the compartment. The introducing means may be in the form of a third magnet.

The apparatus may be one in which the reflective surface is an air:water reflective surface, a mirrored reflective surface, or a waveguide.

The apparatus may be one in which the concentrate is transported in the fluid in the form of the relative motion of the contact between the exterior wall of the sample holder and a second magnet, and including imaging optics for the detection of the analyte.

The apparatus may include multiple compartments for different analytes, with each compartment having a different fluid.

The apparatus may include a second magnet to introduce particles. The fluid contact may be one in the form of one pole of the second magnet, or in the form of a cap of a sample holder, or in the form of a dipstick, or in the form of a swab, or in the form of a syringe. The apparatus may alternatively employ other ways of introducing the second magnet to the compartment.

In a further non-limiting embodiment of the present invention there is provided a method for detecting an analyte using a fluid having magnetic particles with a binding ligand for binding the analyte, which method comprises:
(i) providing the fluid;
(ii) contacting the fluid with the compartment;
(iii) putting the fluid between the first magnet and the optically transparent part of the wall of the compartment;
(iv) transporting the magnetic particles with the binding ligand into the imaging plane, using the first magnet to transport through the fluid and locate in the imaging plane; and
(v) arranging the imaging plane detected to comprise light scattered by the magnetic particles with the binding ligand.

The method of the present invention may be one in which the wall of the compartment is rendered reflective at the plane, illuminated and sized, thereby reflecting the illumination to the location in the imaging plane, i.e. distant from the detector.

The method may include providing a second magnet which provides a reflective surface, contacting the fluid with the second magnet, and collecting the magnetic binding ligand and bonded analyte on the second magnet.

The method may be one in which the reflective surface is part of a wall of the compartment. In this case, the reflective surface may be an air:water reflective surface, a mirrored surface, or a wave guide. In the method of the present invention, other types of reflective surface may be employed.

The method may be one in which the first magnet is a stationary magnet. Alternatively, the method may be one in which the first magnetic is a moveable magnet.

The method of the present invention may include providing imaging optics for the detection of the analyte.

The method may be one wherein there is a second magnet, and where one pole of the second magnet collects the binding ligand.

The method may be one in which the fluid is a gas or a liquid. When the fluid is a liquid, then the liquid may include a second binding ligand with a different specific affinity for the analyte.

The magnet binding ligand and the second binding ligand may have different specific affinities. The magnetic binding ligand and the second binding ligand may have the same specific affinity.

The binding ligand may be, for example, an antibody, a drug, a peptide, or a nucleic acid.

The method may include contacting the fluid with a plurality of magnetic binding ligands with specific affinity for different analytes in the fluid, and contacting the fluid with a plurality of different “labelled” binding ligands with specific affinity for different analytes.

The apparatus and the method of the present invention may be used wherein the fluid is a sample and the sample is food, beverage, bodily fluid or an environmental sample.

The analyte may be a protein, a nucleic acid, a virus, a bacterial cell, a spore, or a fungus cell. Other types of analytes may be employed.

The present invention is able to provide a portable apparatus and a low cost method requiring a low level of technical skill to assay a sample at the point of care or in the field or in the factory, typically for microbiological, immunological and nucleic acid analytes, with no need for automation using moving parts or fluid flow. The sample may be mixed with mobile particles, such as magnetic particles, which bind analytes and any labels, in a chamber where turbulent mixing takes place to extract the analytes. The particles may be transported into an imaging chamber, such as super-paramagnetic particles attracted towards a magnet positioned at the distal end of the imaging chamber, washing the particles and avoiding significant ingress of sample and flow of fluid in the imaging chamber. Particles transporting across the imaged chamber may be tracked and enumerated, to distinguish different types of mobile particles and particles moving randomly by Brownian diffusion only. The imaging chamber may act as a liquid light guide or may be provided with a reflective surface to detect simultaneously light scattering and luminescence of the particles contrasted in a dark field image with little or no optical magnification, relaxing the need for optical lenses, filters and simplifying manufacture. The apparatus may be attached or integrated into a mobile telecommunications device to record and receive information.

The sample may be mixed in a first chamber of the apparatus with mobile particles, such as magnetic particles and typically super-paramagnetic particles, with binding specificity for at least one analyte, together with any reagents to label the analyte and perform the desired assay. Turbulent mixing in the first chamber recovers sample from the sampling device, such as from a swab or syringe, and extracts analyte from the sample bound to the particles. The turbulent mixing produces air bubbles and turbidity from dispersion of air and other particles in the sample. Means may to be provided for imaging to detect and enumerate the mobile particles substantially free of the turbidity in the sample that would otherwise prevent imaging from tracking and enumerating the particles.

The first mixing chamber of the apparatus may be connected to a second imaging chamber via an interconnecting port. The second imaging chamber may be pre-filled by capillary action from the interconnecting port with air backpressure equilibrated from a second port at the distal end of the imaging chamber. Alternatively, the second imaging chamber may also be filled via this second port, including by a different assay medium from that separately charged into the first mixing chamber. In both cases, the second port in the second imaging chamber may then be sealed, prior to sample addition. The sealed second port then avoids turbulent mixing in the first chamber from reaching the region of the second chamber where imaging takes place, leaving the fluid substantially stationary. Turbulence in the first chamber passes across the face of the interconnecting port to the second imaging chamber, which would act to pull fluid from the second imaging chamber to mix in the first imaging chamber. However, the resulting reduced pressure in the sealed second port also tends to prevent flow of fluid across the interconnecting port between the two chambers. Turbulence in the region of the interconnecting port may be further reduced by baffles, including beads or washers, positioned to provide resistance and dissipate flow without blocking particle transport through the interconnecting port. The second imaging chamber thereby remains substantially stationary and free of particle contamination from the first mixing chamber during imaging of the transported mobile particles. Although other particles may diffuse through the interconnecting port between the chambers, this is relatively slow compared to the transport of mobile particles. The second imaging chamber is thereby suitable for tracking and enumeration of mobile particles transported into the second imaging chamber.

As a consequence of using a means of avoiding flow between the first and second chamber, for example as described above, the substantive transport from the first mixing chamber into the second imaging chamber is of mobile particles, such as magnetic particles attracted by a magnet positioned proximal to the imaging chamber wall and distal from the port to the imaging chamber. The relative field gradient proximal to the interconnecting port into the second chamber may be increased by reducing the field strength straying elsewhere in the first chamber by using screens shaped from Mu metal, typically annealed nickel-iron alloy, without affecting the field strength and gradient applied across the second chamber. Such means provides for the analyte particles to pass through the interconnecting port and to wash in the medium in the second imaging chamber proximal to the interconnecting port from the first imaging chamber. Depending upon the size of the particles, the strength and gradient of the field, and size of the second chamber, mobile particles may transport into the second chamber over a period of minutes. Consequently, the mobile particles pass across the second imaging chamber ahead of other sample particles, and collect in the imaging chamber, such as on the wall proximal to a magnet positioned distal to the interconnecting port, between the chambers of the apparatus.

Low numbers of magnetic particles may be enumerated when collected proximal to the magnet. However, the number of magnetic particles used may be greater than the number of particles visible individually in the volume imaged proximal to the magnet. The image (eg 4-12 mm largest dimension) may include the imaging chamber wall proximal to a small magnet (eg 1-4 mm largest dimension) and the medium in the region of the imaging chamber between the washing region, proximal to the interconnecting port, and the magnetic bead collection region, proximal to the location of the magnet. This provides for the mobile particles to be tracked by image analysis algorithms and enumerated during transport to the collection area. The next means may provide for the mobile particles and their luminescent labelling to be visible individually in the image to such particle tracking and enumeration.

The apparatus may provide for imaging from a dark background in order to maximise the contrast of the light scattered by the mobile particles and luminescence from labels within or bound to the particles, and to minimise the optical components and associated manufacture tolerances. The second imaging chamber may act as a liquid light guide to maximise illumination of the imaging chamber from light sources such as LEDs. Light may be launched within the relatively wide acceptance angle of the liquid light guide, relaxing manufacture tolerances, such that illumination light is constrained substantially to the imaging chamber by internal reflection at the walls of the imaging chamber. The intensity of light guided in the imaging chamber and the concentration of fluorophores associated with the particles may provide lasing conditions within the particles contained in the liquid light guide and greater contrast of the mobile analyte particles.

The contrast of the light scattered or emitted from luminescence by the analyte particles may be further enhanced by the illuminating light remaining substantially in the liquid light-guided imaging chamber, to produce a dark background in the volume imaged by the sensor. Light scattered or emitted by particles in the imaging chamber orthogonal to the guided illumination is not substantially reflected within the imaging chamber liquid light guide, and is contrasted against the dark background visible to the imaging sensor. Scattered light may be distinguished from emission of luminescent light from different fluorescent labels by detection of colour using a RGB CMOS imaging sensor, for example, detecting scattering of blue illumination light in the B channel and detection of red and green luminescence in the R and G channels of the imaging sensor. In this way, the need is removed for filters with cut-off wavelength to remove light of the Illumination wavelength, when both scattered light and luminescence of the particles are visible in the apparatus of the present invention.

Consequently, optical components used in the apparatus of the present invention may be simplified to avoid filters to remove illumination light from the image, and to avoid lenses used in light collimating and objective optics. The apparatus of the present invention may be such that it images the particles as objects distant from the imaging sensor. An embodiment of the apparatus in its simplest form may comprise: a light source, such as a LED illuminating the liquid light guide imaging chamber containing the assay medium, and an imaging sensor with RGB pixelation without optical magnification. Additional optical components may be added such as fibre optic coupling between the light source and liquid light guide, an objective lens to vary the image size on the image sensor, and a patterned polaroid filter. The comparatively small number of components, all mass manufactured, provides for low cost apparatus, with relatively relaxed design tolerances facilitating low cost assembly and mass manufacture.

More complex optical arrangements can be used in the laboratory to produce dark field images. The loss of light scattered by particles may be helpful for microscopy, but prevents use of light scattering to detect particles without bound analyte and luminescent label, important in assay applications. The apparatus of the present invention may provide a dark field with contrast of both scattered and luminescent light from the analyte particles, substantially by use of the liquid light guide. However, in the apparatus, illumination with polarised light may also be used to track and enumerate luminescent and scattering particles, by placement of a polaroid filter near orthogonal (eg 70-80°) to the plane of polarisation of the illumination. Light scattered by the particles is substantially removed, but luminescent light is allowed to pass to the imaging sensor. This arrangement may be used when the higher spatial resolution of a black and white imaging sensor (otherwise not distinguishing luminescent and scattered light) is helpful for small particles with relatively low luminescence or when all the RGB channels are used to distinguish multiple luminescent labels. In such cases, a filter with a pattern of polaroid and non-polaroid regions may be used in order to detect scattered light blocked by the polaroid regions, alongside luminescent light passed by both regions of the filter, as particles are tracked through the volume imaged. This means may also be used to distinguish optically active analytes, such as nucleic acids.

Multiple means to distinguish analyte particles may be useful in a variety of assays in the field or factory, or at the point of care, including to perform multiple assays from the same sample, by two or more of the following: to distinguish mobile particles from diffusing particles by their different motion and tracking; particles un-occupied by analyte and label by their light scattering, from particles occupied by analyte and luminescent label; and to detect optically active analytes or label interactions; and to distinguish multiple colours of luminescence.

The apparatus of the present invention may track and enumerate individual particles, providing low detection limits from as few as 1-100 analyte particles or molecules over a range up to billions of analyte particles, such as microbial cells, viral particles, antigen or nucleic acid molecules. In the case of immunoassays and nucleic acid assays, detection over a wide range of binding site occupancy may provide assays in the range from 1-100 molecules in 10⁹, approaching the detection limits of the highest affinity binding sites. The apparatus and method may provide for digital counting of the particulate binding sites enumerated with and without bound analyte, such that assays may have a wide operating range of many orders of magnitude and a detection limit dependent on the number of binding sites per particle (eg 1-100) and number of particles (eg up to 10⁹). As the assay is able to be digital and the particles are able to be contrasted from the background assay medium, variations in background or blank or control levels are of less or no concern, and do not constrain the detection limits of assays according to the apparatus and method of the present invention.

Embodiments of the invention will now be described solely by way of example and with reference to the accompanying drawings in which;

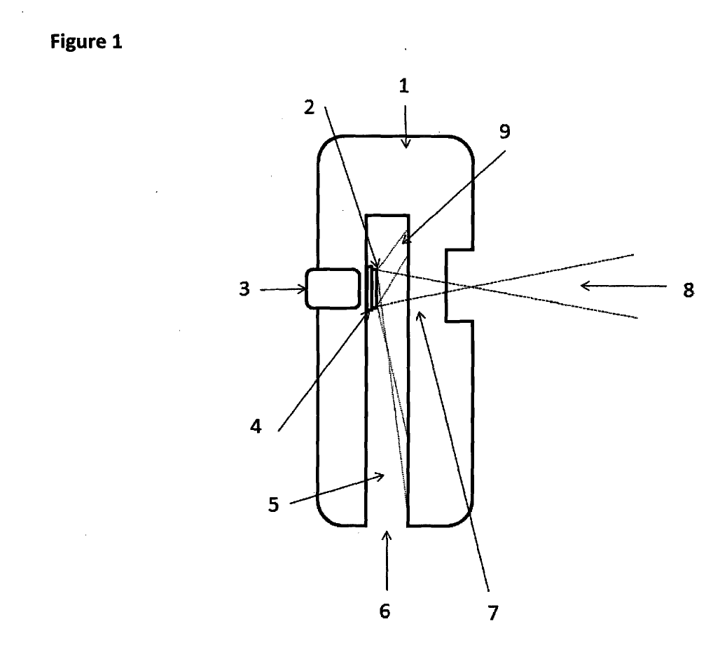

FIG. 1 is a schematic diagram of apparatus of the present invention showing features of a compartment with a first magnet locating particles at the imaging plane to detect analyte with illumination reflecting distant from the detection;

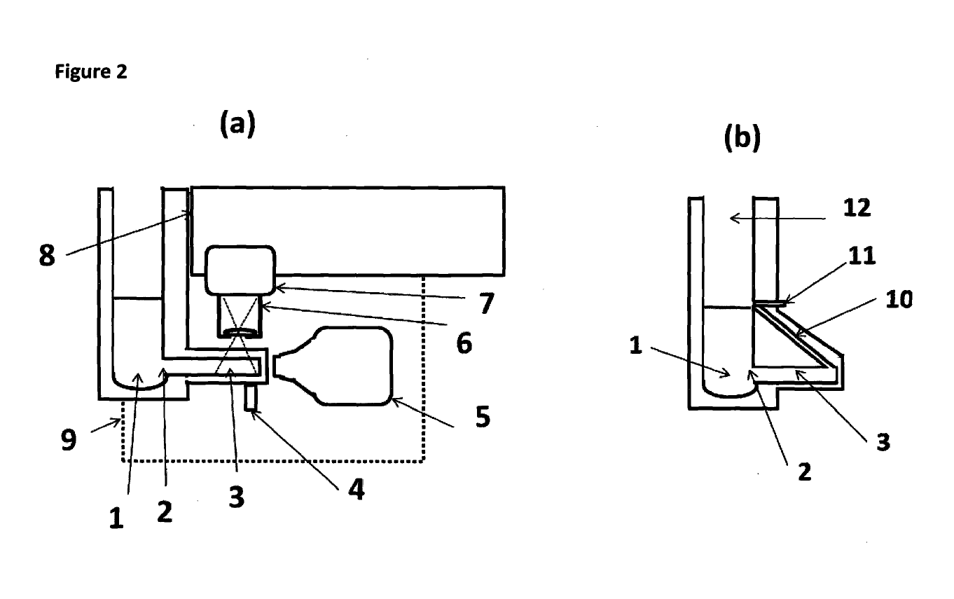

FIG. 2a is a schematic diagram of apparatus of the present invention with a two compartment cartridge attached to a wireless communication device;

FIG. 2b is a side view of the apparatus shown in FIG. 2a;

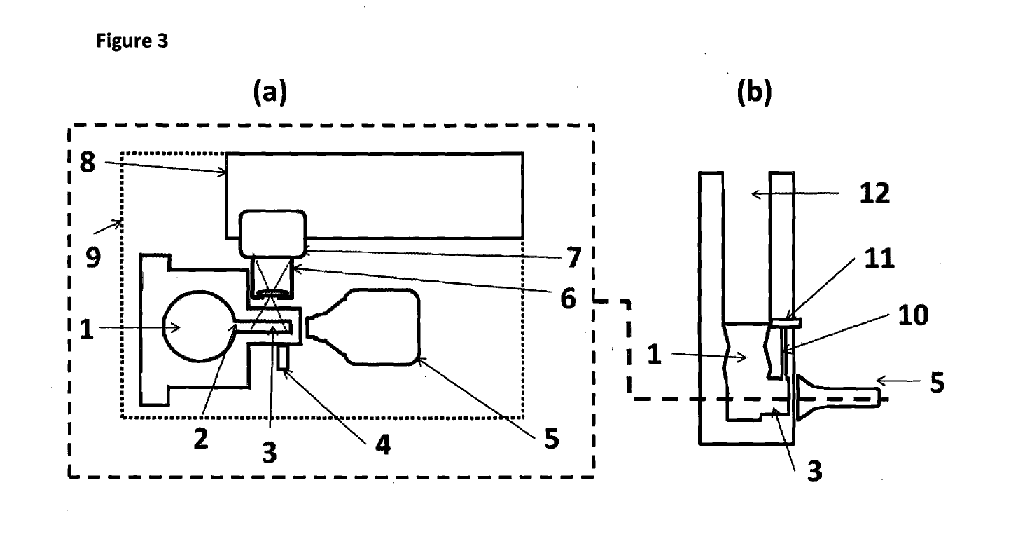

FIG. 3a is a schematic diagram of typical apparatus showing a two compartment cartridge with an in-built wireless communication device;

FIG. 3b is a section through part of the apparatus shown in FIG. 3a;

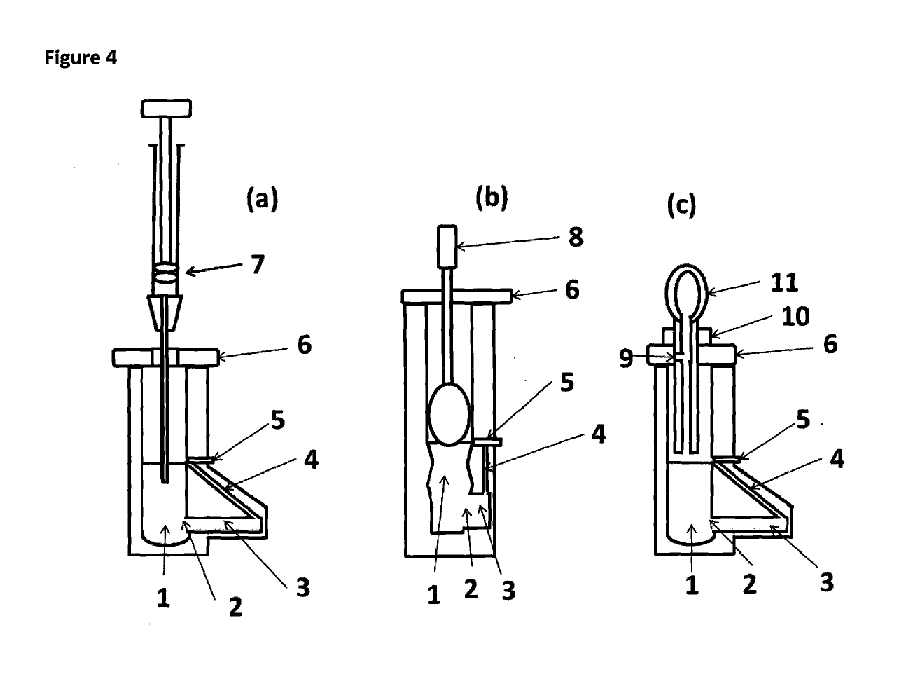

FIG. 4a shows a typical two cartridge arrangement with a sampling device;

FIG. 4b shows a typical two cartridge arrangement with a different sampling device to that shown in FIG. 4a;

FIG. 4c shows a typical two cartridge arrangement with a different sampling device to that shown in FIGS. 4a and 4b;

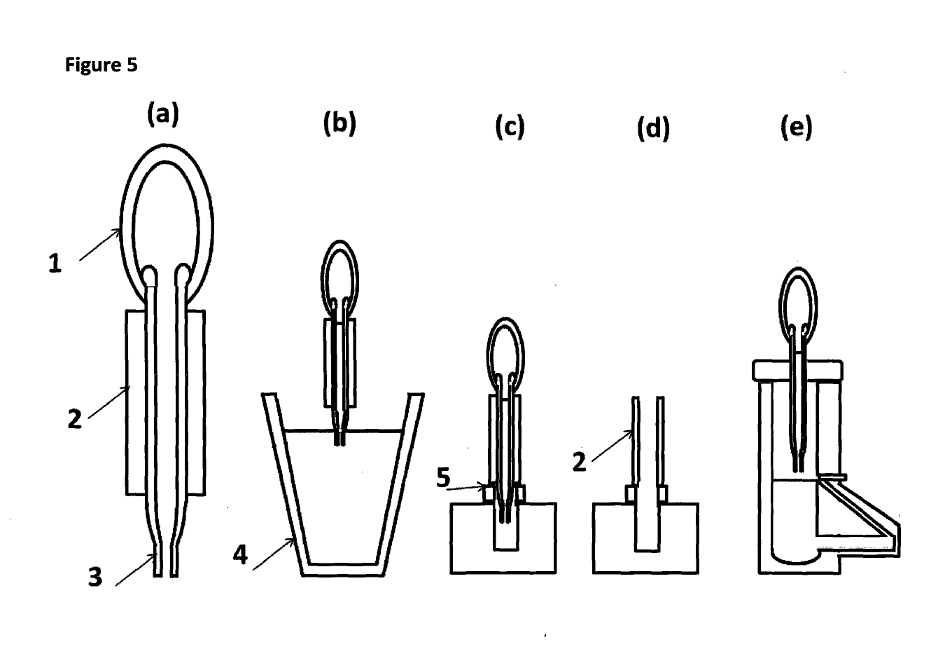

FIGS. 5a-e illustrate the concentrating of a fluid sample from a larger volume of fluid;

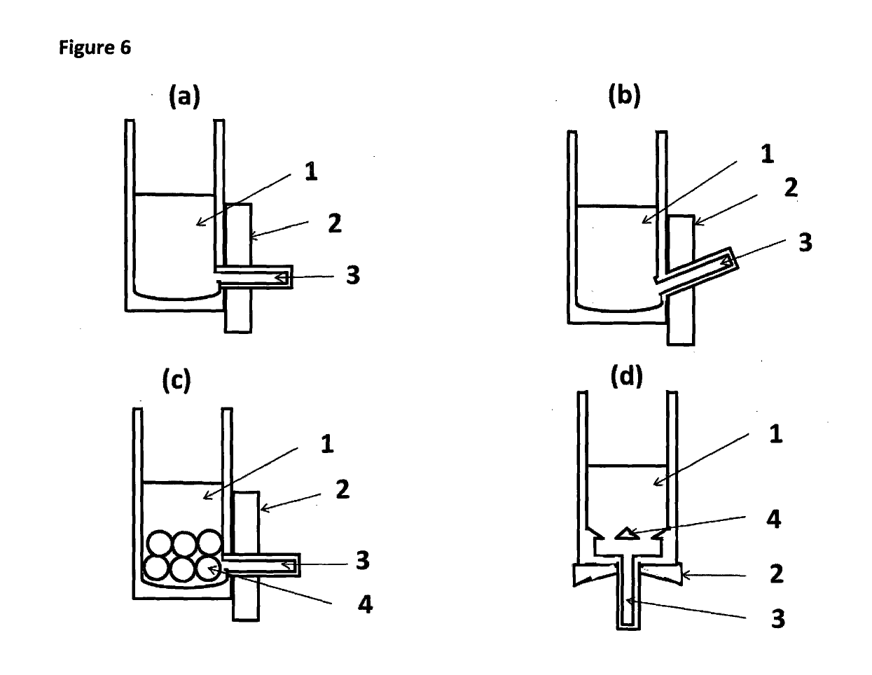

FIGS. 6a-d show examples of different two compartment cartridge arrangements;

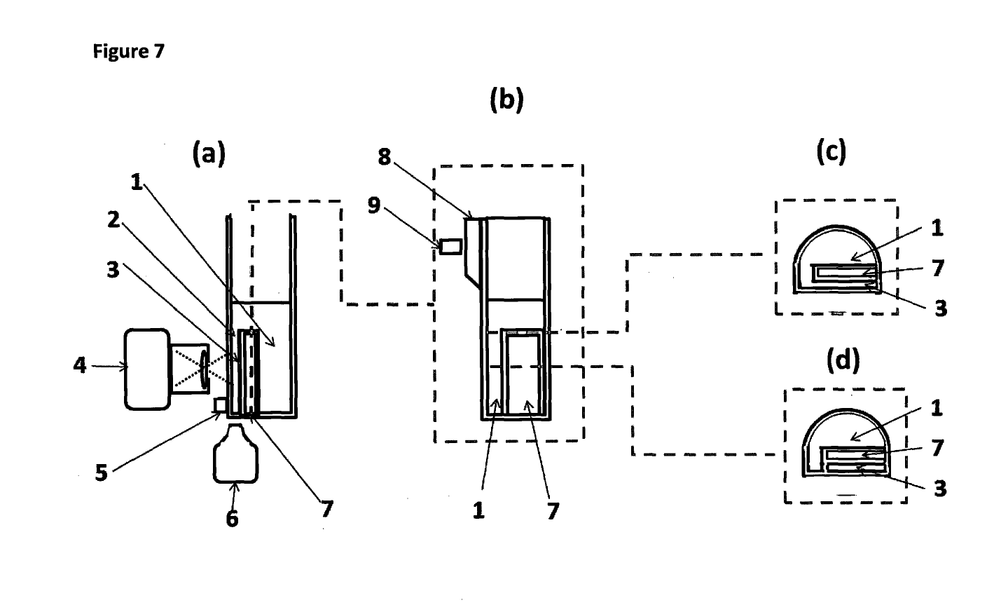

FIGS. 7a-d shows a two compartment cartridge with the compartment within the sample;

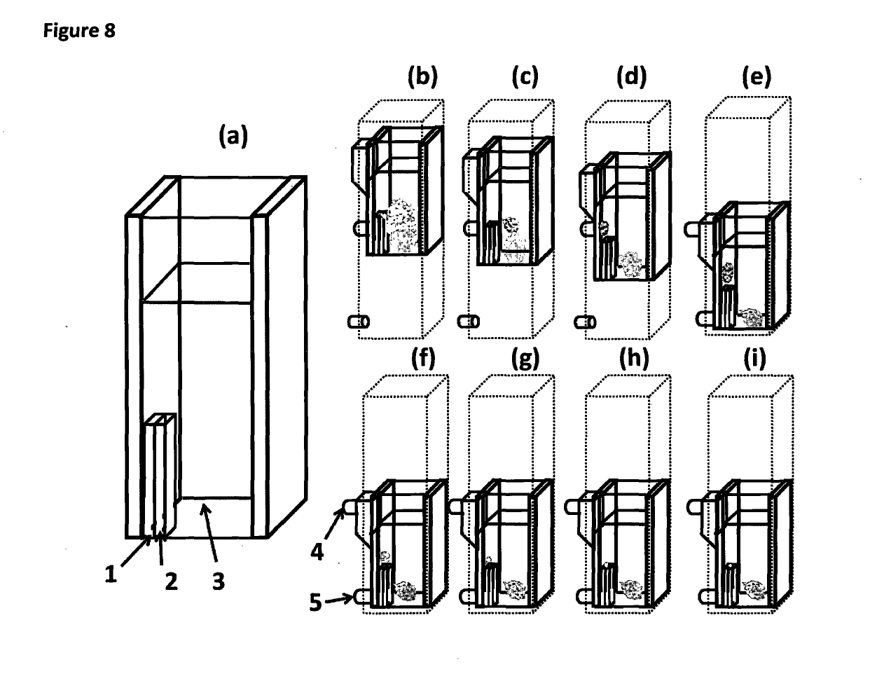

FIGS. 8a-i show the magnetic transport of particles from a sample to a compartment;

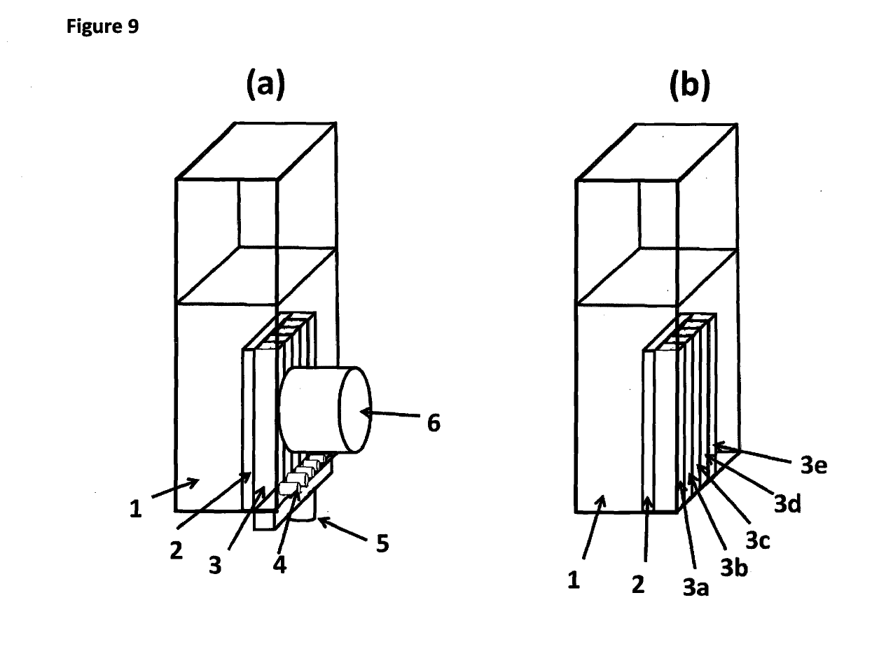

FIG. 9a shows a multiple assay cartridge with five compartments within a sample;

FIG. 9b is like FIG. 9a but omits some parts for ease of illustration;

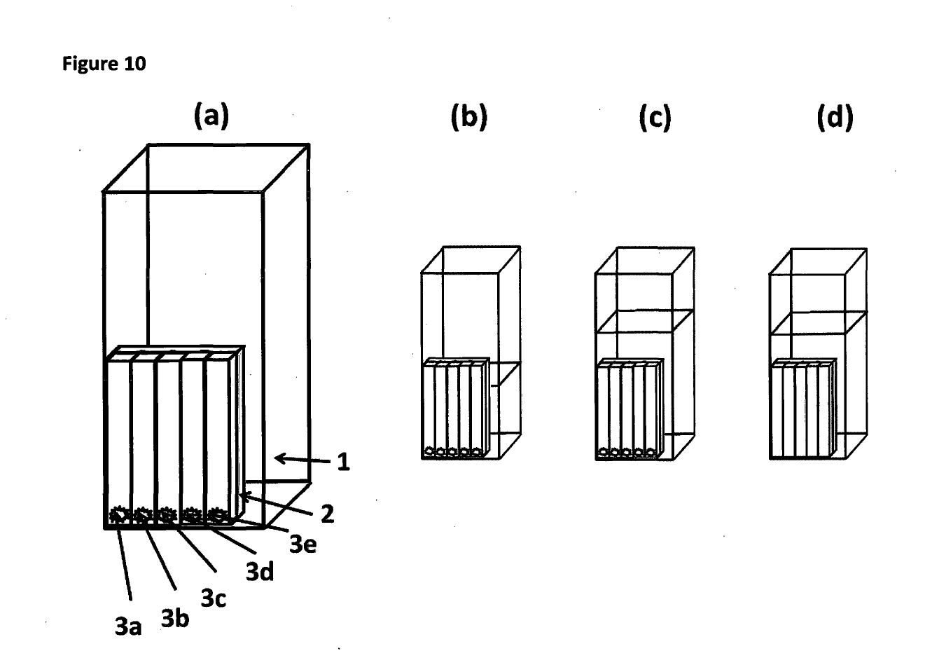

FIGS. 10a-d show a multiple assay cartridge with different reagent in each of five compartments within the sample;

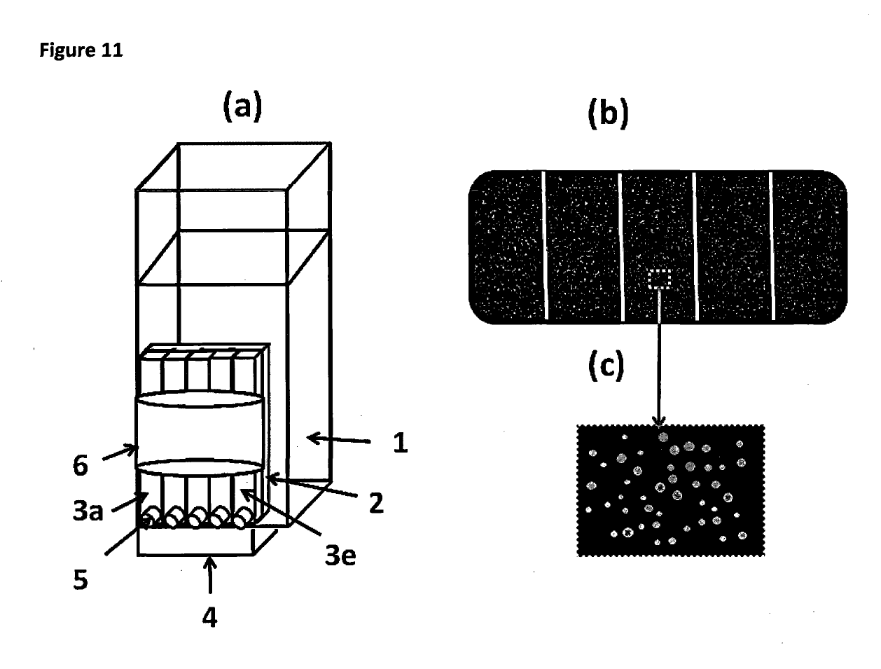

FIGS. 11a-c show a multiple assay cartridge with five different assay media and multiple assays in each assay medium;

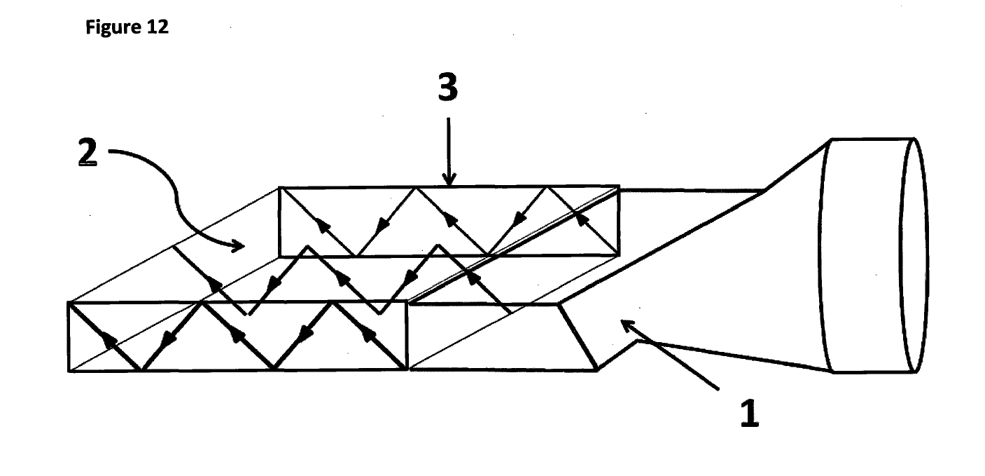

FIG. 12 is a schematic illustration of a second compartment of the apparatus of the present invention as a light guide using a fishtail coupling from a light source;

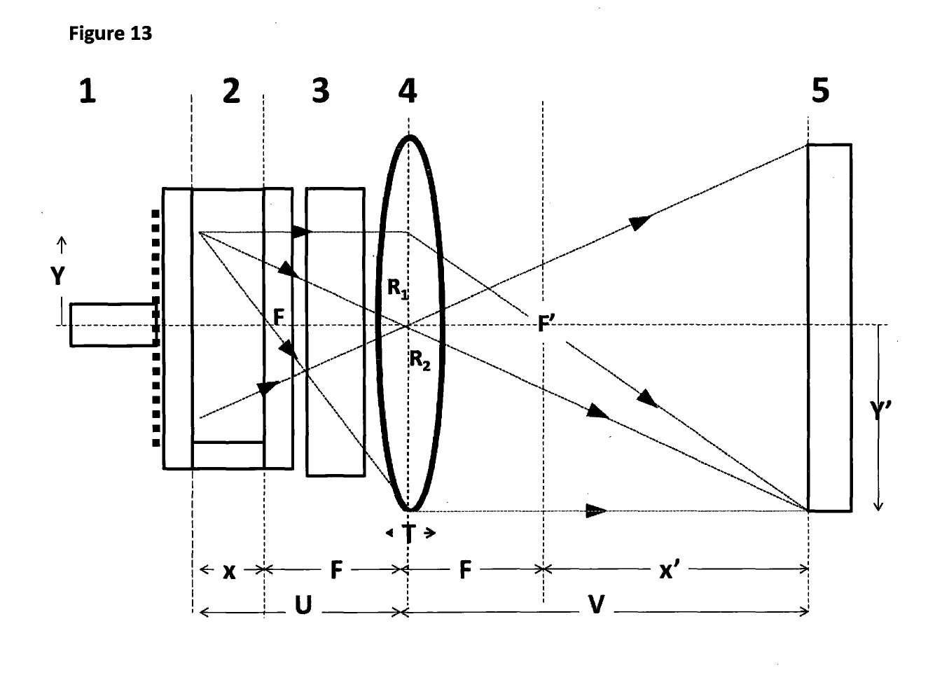

FIG. 13 shows an optical layout for imaging means of the apparatus of the present invention;

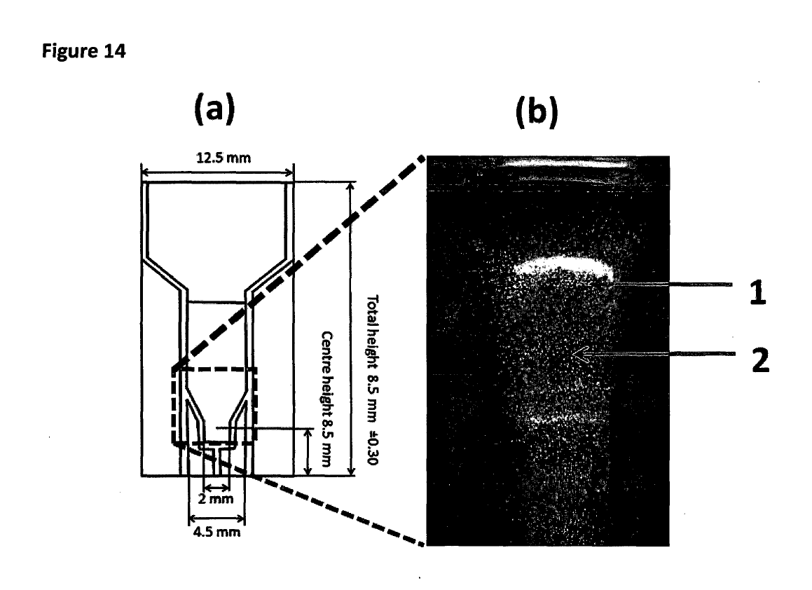

FIGS. 14a and 14b show collection of magnetic particles in a sampling compartment;

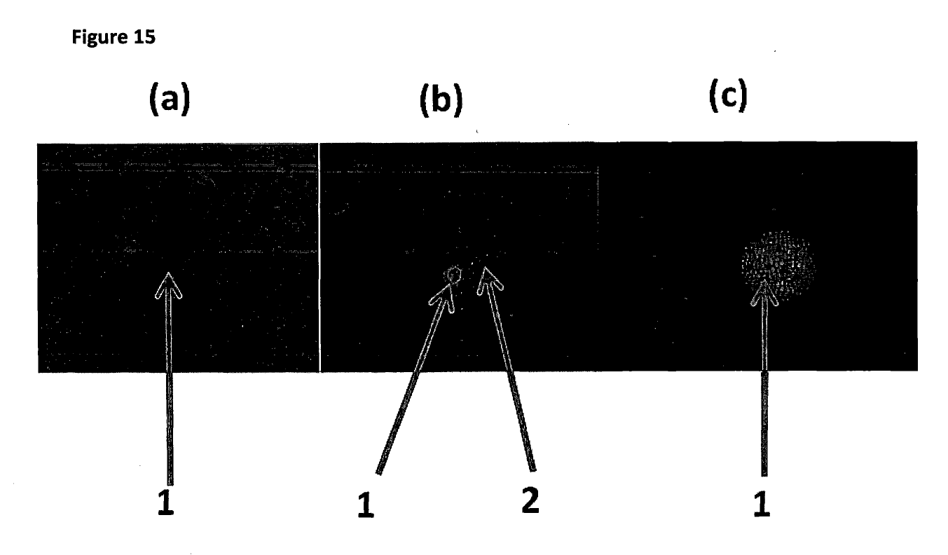

FIGS. 15a-c show magnetic particle transport of the plane of imaging in a compartment;

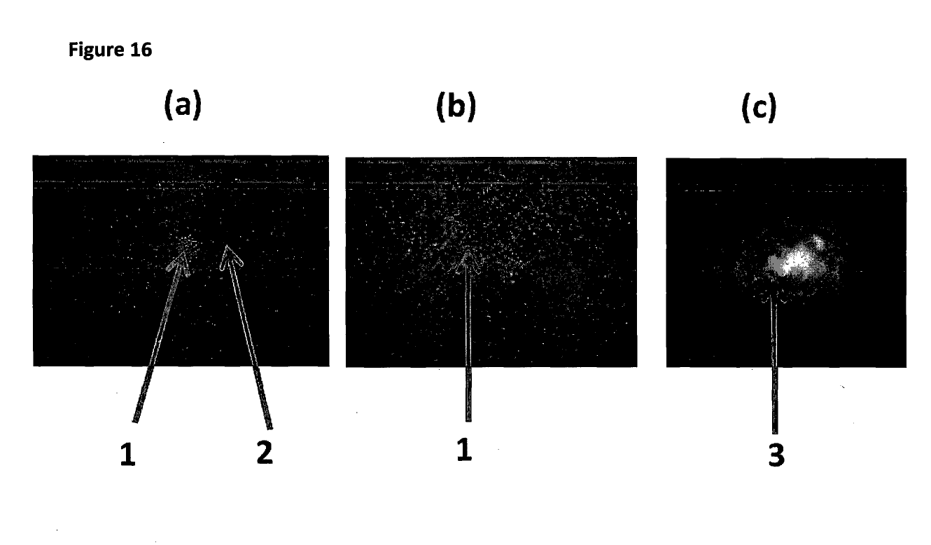

FIGS. 16a-c show an assay of Clostridium difficile spores;

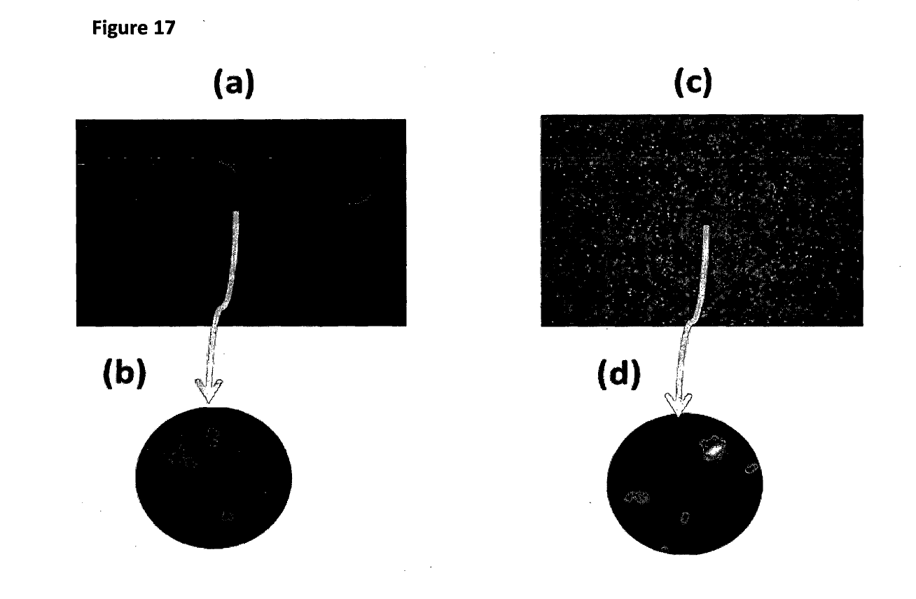

FIGS. 17a-d show images of an immunoassay using protein G beads;

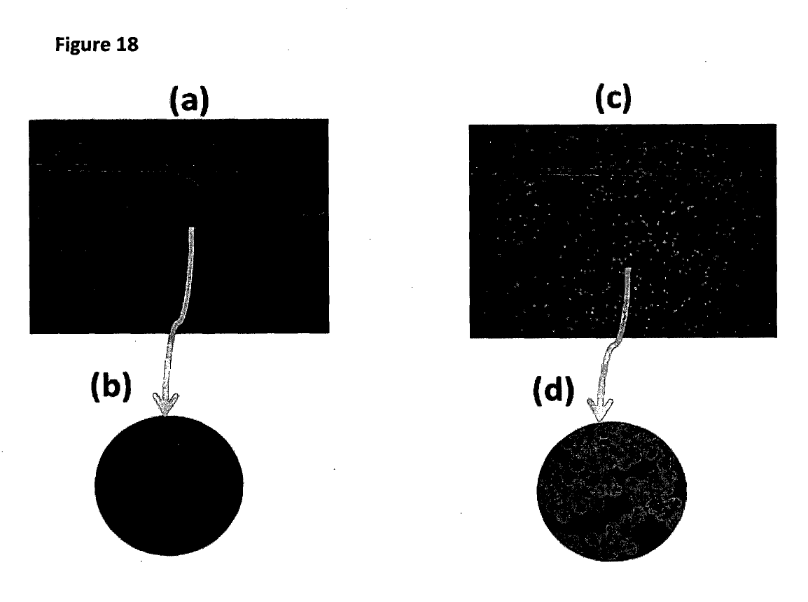

FIGS. 18a-d show images of an immunoassay using streptavidin beads;

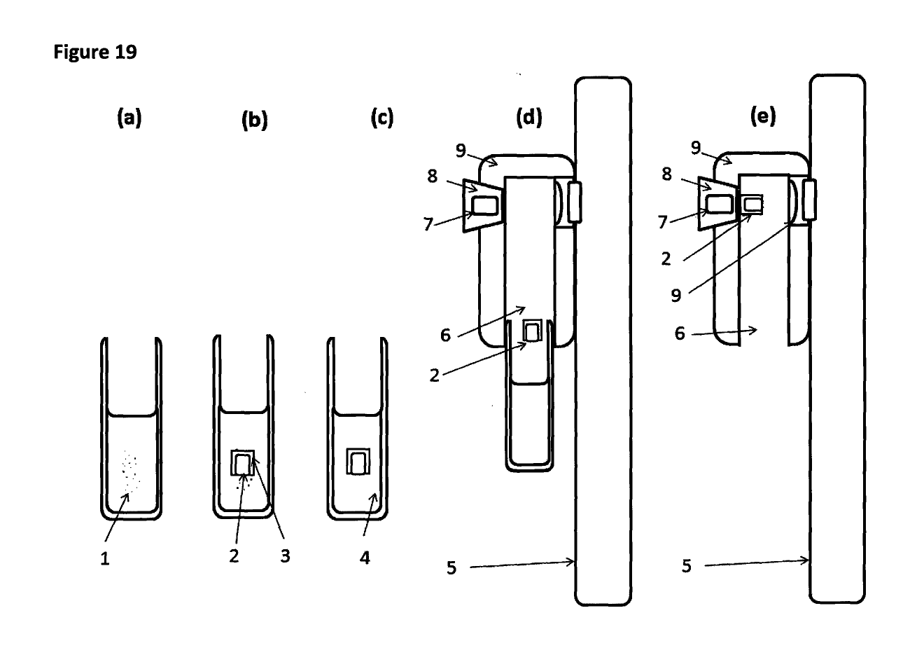

FIG. 19a-e shows the collection of magnetic particles from a sample with a second magnet transporting through the compartment;

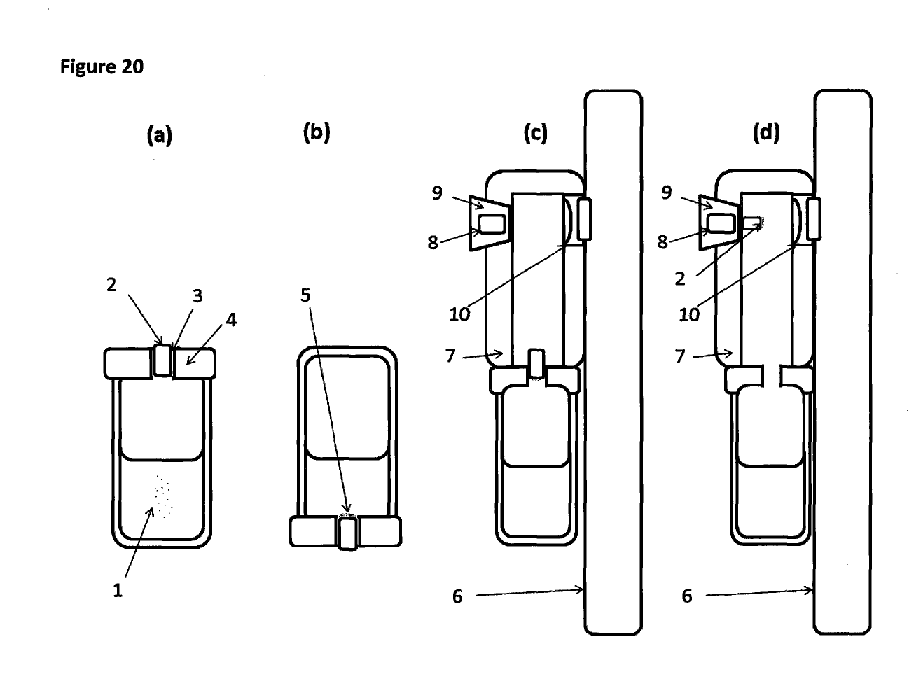

FIG. 20a-d show collection of magnetic particles from a sample with a second magnet in a cap of a sample container;

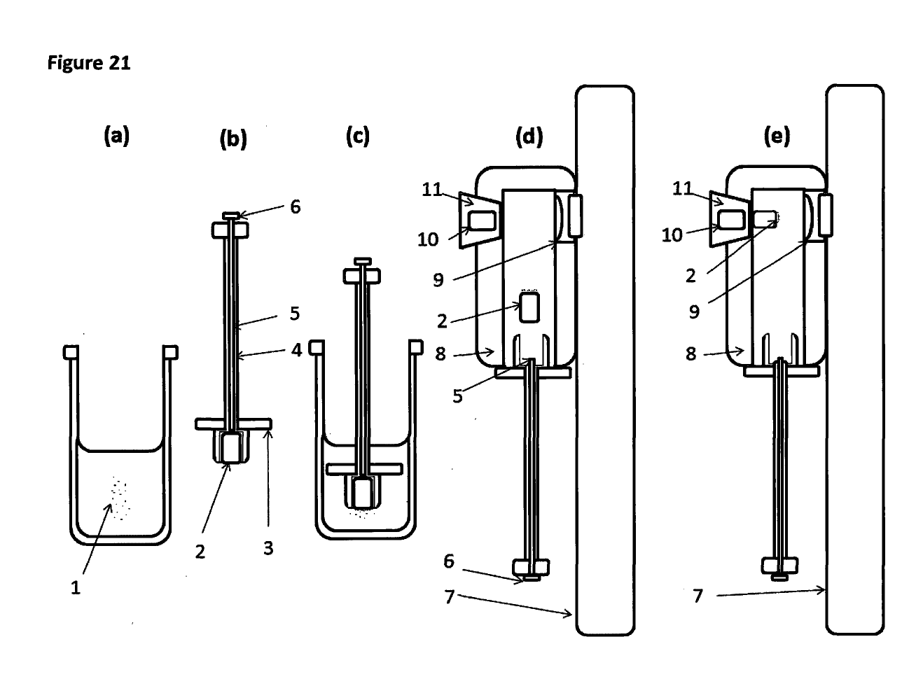

FIGS. 21a-e show collection of magnetic particles from a sample with a second magnet in a housing on a dipstick;

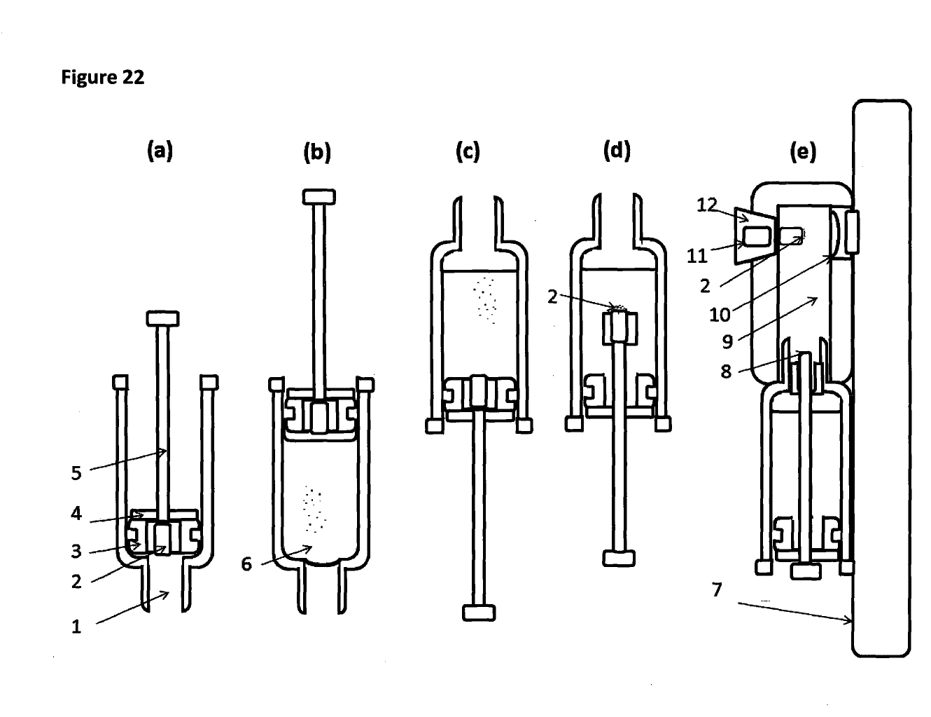

FIGS. 22a-e show collection of magnetic particles from a sample on a second magnet in a housing in the seal of a plunger in a syringe;

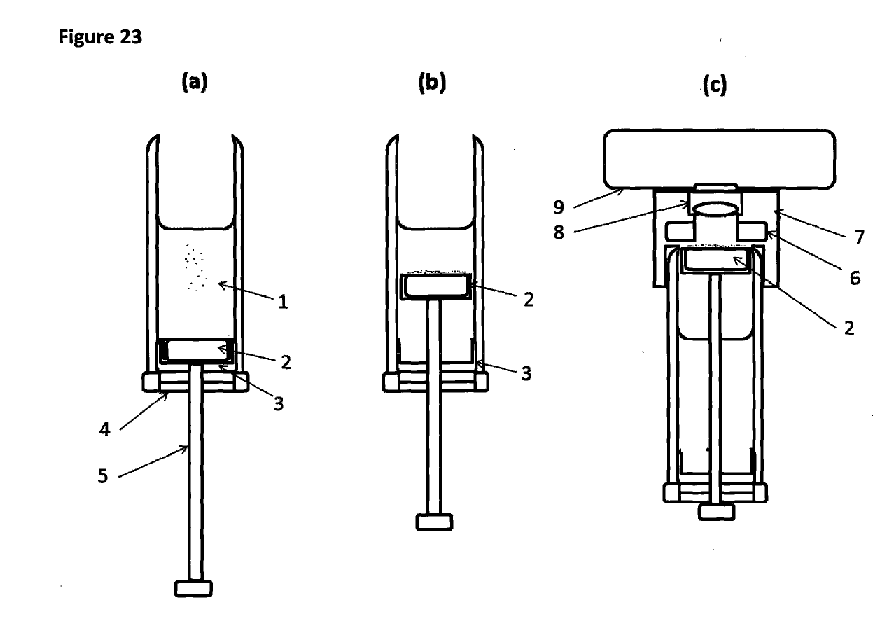

FIGS. 23a-c show collection of magnetic particles from a sample on a second magnet in a housing in a seal of a plunger contacting a toroidal magnet in a compartment;

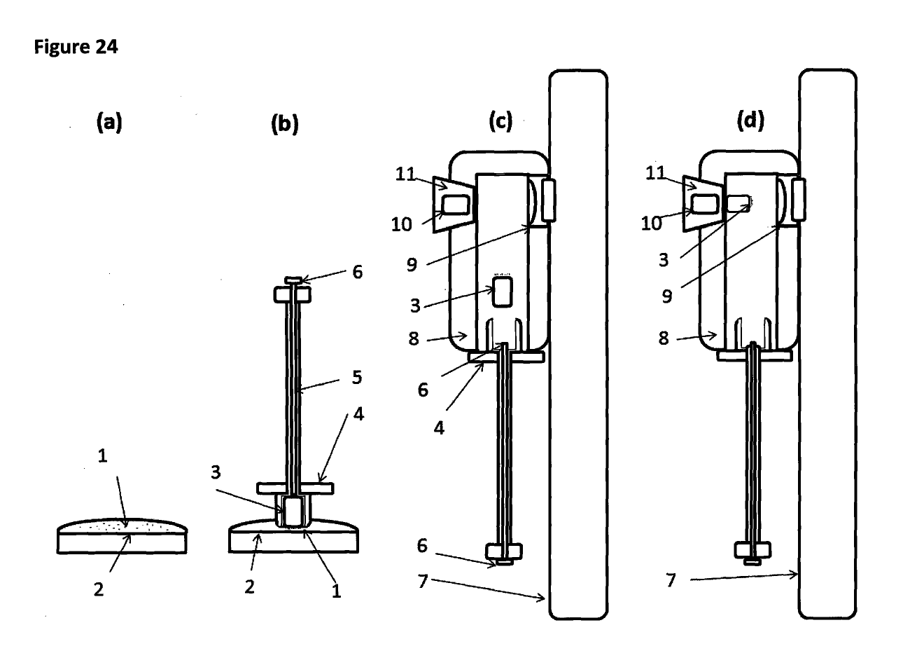

FIGS. 24a-d show collection of magnetic particles from a sample using a magnetic swab;

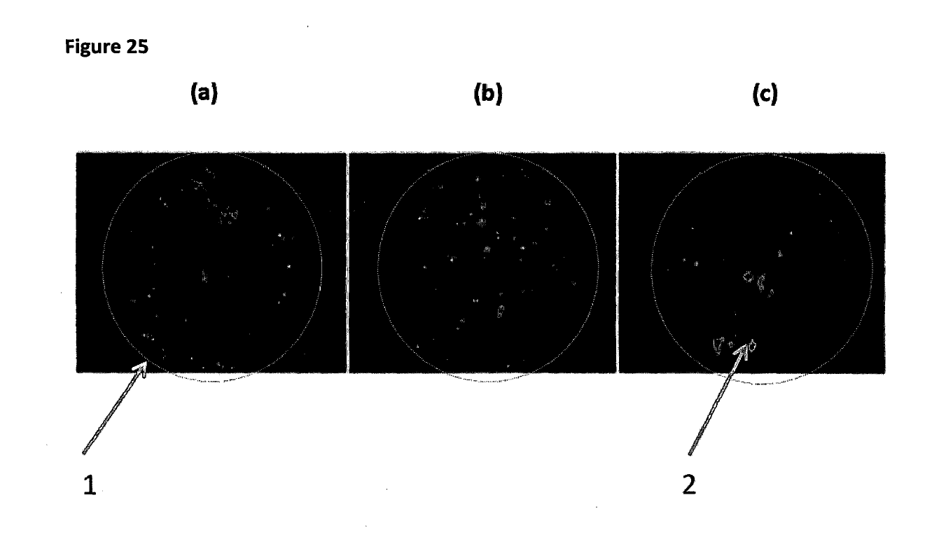

FIGS. 25a-c show detection of Clostridium difficile spores swabbed from a surface; and

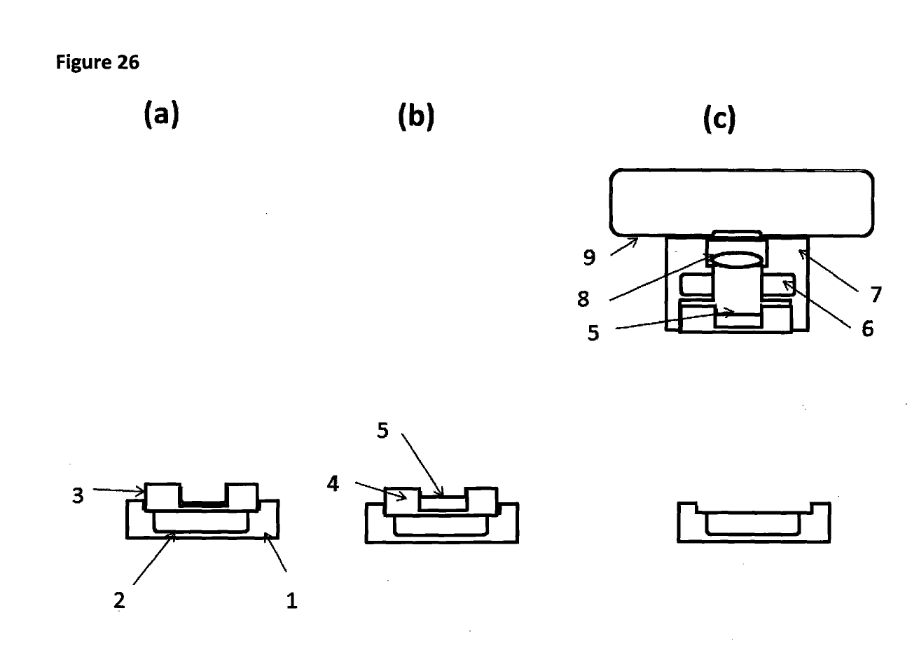

FIGS. 26a-c show collection of magnetic particles in a well with an air/liquid interface providing a reflective surface.

FIG. 1 illustrates a schematic layout of features of a compartment 1 containing a fluid 5 with a port 6 to receive and to locate magnetically captured particles 2 onto an illuminated 9 reflective surface 4 at the plane of imaging 8 by using a first magnet 3 in contact with the outer wall of the compartment in order to detect the analyte through a transparent wall 7 of the compartment.

FIGS. 2a and 2b illustrate a schematic layout of typical two chamber cartridge apparatus attaching to a mobile wireless communications device 8. More specifically, FIG. 2a shows a vertical section through the central region of the apparatus illustrating the imaging arrangements and FIG. 2b shows a different vertical section through the side of the apparatus showing the fluid arrangements. A first chamber 1 is connected through an interconnecting port 2 to a second chamber 3. The second chamber 3 is filled with assay medium either through the interconnecting port 2 from the first chamber 1 with backpressure equilibrated through a port 10 in the second chamber 3, or filled with different assay medium from the port 10 in the second chamber 3. In both cases, the port 10 in the second chamber 3 is then sealed 11 before addition of a sample. The sample is added to the first chamber 1 by a sampling device such as a swab or syringe or pipette (illustrated in FIG. 4) through a port 12 to the first chamber 1. Particles mixed with the sample in the first chamber 1 extract analyte and transport through the interconnecting port 2 by a field applied to the second chamber 3. In this example, the field is applied by a permanent magnet 4 positioned proximal to the second chamber 3 wall, here distal from the interconnecting port 2, The second chamber 3 is illuminated by a light source 5, such as a tight-emitting diode (LED), here through a window (see FIG. 12) at the end of the second chamber 3 distal from the interconnecting port 2. Particles transporting into the second chamber 3 are imaged through an objective assembly 6 (see FIG. 13) by an image sensor 7 connected to a wireless telecommunications device 8 via an attachment port in the case of the apparatus, and indicated in dotted outline position 9 for simplicity.

FIG. 3 illustrates a schematic layout of typical apparatus with an in-built mobile wireless communications device 8. FIG. 3a is a horizontal section through the apparatus illustrating the imaging layout with a dotted line showing the position of the section illustrated in FIG. 3b of a vertical section through the apparatus omitting the imaging arrangements except for the light source 5. The first chamber 1 is connected through an interconnecting port 2 to the second chamber 3. The second chamber 3 is filled with assay medium either through the interconnecting port 2 from the first chamber 1 with backpressure equilibrated through a port 10 in the second chamber 3 or filled with different assay medium from the port 8 in the second chamber 3. In both cases, the port 10 in the second chamber 3 is then sealed 11 before addition of a sample. The sample is added to the first chamber 1 by a sampling device such as a swab or syringe or pipette illustrated in further examples below, and in FIG. 3 through a port 12 to the first chamber 1. Particles mixed with the sample in the first chamber 1 extract analyte and transport through the interconnecting port 2 by a field applied to the second chamber 3. In this example, the field is applied by a magnet 4 positioned proximal to the second chamber 3 wall, distal from the interconnecting port 2. The second chamber is illuminated by a light source 5, such as a light-emitting diode (LED), through a window further described in FIG. 12 at the end of the second chamber 3 distal from the interconnecting port 2. Particles transporting into the second chamber are imaged through an objective assembly 6 (further described in FIG. 13) by an image sensor 7 connected to a wireless telecommunications device 8 within the case of the apparatus, and indicated in dotted outline position 9 for simplicity.

FIGS. 4a-c schematically illustrate two chamber cartridge arrangements for use with different sampling devices (from left to right). FIG. 4a shows syringe 7 needle injection and mixing in the medium in the first chamber 1 through a seal in the cartridge cap 6. FIG. 4b shows swab 8 extraction into the medium in the first chamber 1 by turbulent mixing by passage across corrugation in the first chamber walls. FIG. 4c shows a pipette sampling a fixed volume of sample by capillary action with air equilibration by the side port 9, expelled into the cartridge by pressure from the bulb 11 maintained by sealing the side port 9 in the cap 6 using the stop 10 to position the side port in the cap. In all cases, analyte is extracted from the sample onto particles in the first chamber 1, which transport through the interconnecting port 2 into the second chamber 3, which is pre-filled with medium using the port 4 in the second chamber and sealed 5.

FIGS. 5a-e schematically illustrate the concentration of analyte from a fluid such as water. FIG. 5a shows a pipette comprising a flexible bulb 1 to fill and empty the pipette 3 is surrounded by a magnet sleeve to capture magnetic particles. FIG. 5b shows the pipette may be repeatedly filled and emptied to sample a larger volume of fluid. Upon each fill of the pipette, by squeezing and releasing the bulb 1 magnetic particles interact with and capture analyte and become recaptured on the wall of the pipette proximal to the magnet sleeve 2, where the magnetic particles remain when the pipette is emptied by squeezing the bulb. Repeated filling and emptying of the pipette circulates a larger volume of the fluid being sampled through the pipette (n×Vp=Vs; where n is the number of fills of the pipette, Vp is the volume of the pipette and Vs is the total volume sampled). FIG. 5c shows the magnet sleeve 2 is removed from the pipette by contacting a station with an additional magnet 5. FIG. 5d shows pulling the pipette from the magnet sleeve, which remains in contact with the additional magnet 5. The magnetic particles inside the pipette are then released and tend to move towards the bottom of the pipette. FIG. 5e shows the magnetic particles may then be charged into the first chamber of the two chamber cartridge by flushing the pipette with assay medium as described for FIG. 4.

FIGS. 6a-d schematically illustrate examples of two chamber cartridges, combining different arrangements of the first chamber 1, magnetic field screen of Mu metal of nickel iron alloy to reduce magnetic field strength and to minimise accumulation of magnetic particles on the walls of the first chamber proximal to the interconnecting port to the second chamber, and baffles 4 to reduce turbulence in the interconnecting port between the first and second chamber. FIG. 6a shows an orthogonal side port second chamber 3 with magnetic field screen 2 to the first chamber 1. FIG. 6b shows an angled side port second chamber 3 with magnetic field screen 2 to the first chamber 1 to avoid sedimenting particles entering the second chamber. FIG. 6c shows wider orthogonal side port second chamber 3 with spherical bead baffles 4 proximal to the interconnecting port between the first 1 and second chamber 3. FIG. 6d shows second chamber 3 with interconnecting port at a base of first chamber 1 with shaped magnetic field screen 2 to increase the magnetic field gradient at the interconnecting port to the second chamber 3, and protected from sedimenting particles and turbulence with a baffle 4 above the interconnecting port.

FIGS. 7a-c schematically illustrate an example of a two chamber cartridge device where the second chamber is incorporated into the first chamber. FIG. 7a shows a vertical section through the device with the cartridge fully inserted showing the first chamber containing the sample 1, the interconnecting port 2 between the first chamber 1 and second chamber 3, the imaging 4 of particles transported through the second chamber by the field applied by a magnet 5, and the illumination of the second chamber by the light source 6. An air-filled chamber 7 alongside the second chamber 3 provides one wall of the liquid light guide of the second chamber 3. The dotted line indicates the position of an orthogonal vertical section FIG. 7c through the air-filled chamber 7 and first chamber 1 showing a magnetic field screen of Mu metal 8 and resting position of a second magnet 9 when the cartridge is fully inserted. Dotted lines show the position of two transverse sections with FIG. 7c being through the top of the air-filled chamber 7 showing the interconnecting port 2 between the first and second chamber, and FIG. 7d being a lower section through the air-filled chamber 7 and the first 1 and second 2 chamber below the position of the interconnecting port. The magnetic particles are collected in the first chamber 1 by a first magnet 9 as the cartridge is inserted into the device, first collecting particles from the bottom of the cartridge and progressively collecting particles from the upper regions of the cartridge as the cartridge is inserted. Prior to full insertion of the cartridge, the first magnet is moved away from contact with the cartridge or to a position behind a Mu metal magnet screen 8 leaving free the collected magnetic particles proximal to the interconnecting port 2 to the second chamber 3. Upon removal of the first magnet from contact with the cartridge, the magnetic particles are released proximal to the interconnecting port when, upon insertion of the cartridge, the magnetic particles are transported into the second chamber by the magnetic field applied by a second magnet 5, where the magnetic particles are illuminated by the light source 6 and imaged 4.

FIGS. 8a-i schematically illustrate magnetic transport in a two chamber cartridge. FIG. 8a shows the second chamber 1 within the first chamber 3 separated by an air-filled chamber. The cartridge is inserted into the reader device and the position on insertion is shown here as a transparent skeletal insertion port for clarity. FIG. 8b shows how magnetic particles mixed with the sample in the first chamber begin to separate in the magnetic field of the first magnet 4. FIG. 8c shows how magnetic particles separate from sample particles as the cartridge inserts. FIG. 8d shows magnetic particles separated from the sample particles and positioned above the port to the second chamber. FIG. 8e shows first magnet 5 moved away from the sample as the cartridge is fully inserted, releasing the magnetic particles collected proximal to the port to the second chamber. FIGS. 8f-i show how magnetic particles transport into the second chamber in the magnetic field of the second magnet 5.

FIGS. 9a-b schematically illustrate a multiple assay cartridge with five second chambers in the first chamber 1. FIG. 9a shows an air-filled chamber 2 and the five second chambers 3 comprising five liquid light guides illuminated by the light source 5, with a magnet 4 applied to each of the second chambers, and an image sensor monitoring the image of the particles transporting through each second chamber towards the magnet. FIG. 9b shows the same cartridge illustrating the five second chamber liquid light guides, from left front 3a to right back 3e.

FIGS. 10a-d show a multiple assay cartridge with a different reagent in each of five second chambers within the first chamber. More specifically, FIG. 10a schematically illustrates a multiple assay cartridge with five different solid reagents from left 3a to right 3e in the five respective second chambers, separated from the first chamber 1 by an air filled chamber 2. FIG. 10b shows the same multiple assay cartridge with sample mixing in the second chamber with the mixed sample fluid level below the entry to the second chambers. FIG. 10c shows the mixed sample fluid above the entry to the second chamber and filling the five second chambers. FIG. 10d shows a different solid reagent dissolved in the fluid filling each of the five second chambers.

FIGS. 11a-c show a multiple assay cartridge with five different assay media and multiple assays in each assay medium. More specifically, FIG. 11a shows a cartridge with five second chambers, from left 3a to right 3e within the first chamber 1 containing sample and separated from the first chamber by an air-filled chamber to form five liquid light guides 3a to 3e illuminated by the light source 4 and imaged by the image sensor 6. Magnetic particles with captured analyte transport from the first chamber into each of the five second chambers in the field of the five magnets 5 applied to each the outer surfaces of the second chambers. Each of the five second chambers has a different assay medium reacting with the analytes captured on the magnetic particles transporting through each of the five second chambers. Referring to FIG. 11b, the image sensor 6 monitors the transport of the magnetic particles reacted with the assay medium in each of the second chambers. Magnetic particles of different size are modified to capture different analytes, and become fluorescent upon reaction with the assay medium in each second chamber, FIG. 11c. At least five different magnet particles sizes with five different captured analytes can be assayed in each second chamber. Within each second chamber, at least three different fluorescent reactions can be assayed for each of the five different magnetic particles and analytes detectable in each assay chamber. Magnetic particles with captured analytes with no fluorescent reactions in the particular second chamber assay medium are not detected, and may be assayed in another second chamber. In a cartridge with five second chambers, twenty five different captured analytes may be assayed, and each analyte may be subjected to at least three fluorescent reactions assays, providing overall at least seventy five different assays of at least twenty five different analytes.

FIG. 12 schematically illustrates illumination of the second chamber of the apparatus as a liquid light guide using a ‘fish-tail’ coupling from the light source. Thus, the light source is provided with a ‘fish-tail’-shaped coupling 1 to launch light into the liquid light guide provided by the second chamber 3 of the cartridge, with interconnecting port 2 to the first chamber of the cartridge not shown. Light is internally reflected at the wall of the image chamber to ambient air and guided in the second chamber liquid light guide, indicated by the optical path ray diagram arrows.

FIG. 13 schematically illustrates a typical optical arrangement and optical ray diagram for imaging particles transporting in the second chamber, here by a magnetic field provided by a magnet 1 applied to the wall of the second chamber 2. The image may be taken directly through the aperture in front of the image sensor 5. Alternatively, as illustrated, the image size Y′ of the object size Y on the image sensor may be adjusted by an objective lens 4 and the image modified optionally by a filter, such as a polaroid filter. The objective lens is typically a bi-convex lens (as shown here) or positive meniscus lens to increase the numerical aperture. The object-to-image distance D is the sum of the object-to-lens U and lens-to-image V distances, comprising the depth of the second imaging chamber x and the focal points F of the lens in the object and image plane and the distance x′ from the image focal point F to the image sensor. The magnification M desired of the objective lens may be calculated (as M=V/U or M=Y′/Y). The focal length f) of the lens required may be calculated (as f=U×M/M+1), including from the radii Ri & R₂) of the respective sides of the lens for z thin lens, as 1/f=n−1)×1/R₁)−1/R₂)), where n is the refractive index of the lens, or by using thick lens formulae).

FIGS. 14a-b illustrate the collection of magnetic particles in the sample chamber. FIG. 14a shows a layout of the sample mixing chamber with dimensions in millimetres. FIG. 14b shows an image taken of upward transport of magnetic particles 2 in the sample chamber for collection at the location 1 for transport into the imaging chamber.

FIGS. 15a-c show images within the imaging chamber of a cartridge with a magnetic disc (Neodymium N42 NdFeB+NiCuNi of a diameter of 1 mm) to transport magnetic particles 2.3 μm diameter Spherotech beads (with green fluorescein fluorescence) across the cartridge imaging chamber to the location of the magnetic disc. FIG. 15a shows before magnetic transport of the particles across the chamber. FIG. 15b shows the transport of magnetic particles, with individual particles visible during transport 2, across the chamber to collect on the chamber wall proximal to the location of the magnet 1. FIG. 15c shows the magnetic particles collected on the wall proximal to the magnet with individual magnetic particles visible and remaining individual magnetic particles also visible during their transport.

FIGS. 16a-c show the assay of Clostridium difficile spores. FIG. 16a shows an image of the transport and collection of C. difficile spores captured on magnetic particles by antibodies against C. difficile spores immobilised on the magnetic particles. The C. difficile spores are treated with a fluorescent staining reagent (Aojula et al. (2012) Method Reagent & Apparatus for Detecting a Chemical Chelator. Int. Pat. Appl. WO 2012/017194 A1). Referring to FIG. 16a, the image shows fluorescent C. difficile spores captured on magnetic particles collecting on the cartridge wall proximal to the location of the magnet 1 and transporting towards the magnet 2 such that the number of magnetic particles with captured spores is enumerated. FIG. 16b shows an image of the transport and collection of C. difficile spores in the region of the cartridge wall proximal to the magnet, showing magnetic particles transporting towards the magnet and magnetic particles captured on the cartridge wall proximal to the magnet 1. FIG. 16c shows an image of a magnetic particle showing the capture of multiple C. difficile spores on the magnetic particle.

FIGS. 17a-d show images of an immunoassay using Protein G beads. FIG. 17a shows magnetic beads (4.1 micron diameter) modified with Protein G used to capture rabbit IgG antibody and an image taken of the magnetic beads collected in the region of the cartridge wall proximal to a magnet 1 mm diameter. Beads were not visible in the apparatus, but could be detected in FIG. 17b the image taken from a microscope. FIG. 17c shows excess fluorescein-labelled goat anti-rabbit IgG added to the rabbit IgG captured on the magnet beads, and imaged. Fluorescently-labelled magnet beads were visible in the image 133,500, in the images from the apparatus. FIG. 17d shows images taken from a microscope.

FIG. 18a shows images of an immunoassay using Strepatvidin beads. Magnetic beads 4.1 micron diameter modified with Streptavidin were used to capture biotin-labelled rabbit IgG antibody and an image taken of the magnetic beads collected in the region of the cartridge wall proximal to a magnet 1 mm diameter. Beads were not visible in the apparatus, but could be detected in FIG. 18b the image taken from a microscope. FIG. 18c shows excess fluorescein-labelled goat anti-rabbit IgG added to the rabbit IgG captured on the magnet beads, and imaged. Fluorescently-labelled magnet beads were visible in the image 13,430 in the images from the apparatus. FIG. 18d shows images taken from a microscope.

FIGS. 19a-e show collection of magnetic particles from a sample with a second magnet transporting through the compartment. FIG. 19a shows how the sample is mixed with magnetic particles and labels with affinity for the analyte 1. The magnetic particles and any bound analyte and label are collected by a second magnet 2 in a housing 3. The housing 3 may provide for the magnetic particles to be collected predominantly on a particular pole of the second magnet 2, which pole has the same magnetic polarity as the pole of the magnet 7 presented to the interior of the compartment 6, such that the magnetic pole in the housing around the second magnet is attracted to the opposite pole of the magnet 7 presented to the interior of the compartment 6. FIG. 19c shows how the magnet may be transferred to a further medium 4 to label the analyte bound to the magnetic particles collected by the magnet. FIG. 19d shows how the second magnet 2 is moved to the port of the compartment 6 attached to a device fitted with a camera, such as a mobile phone or smartphone 5, and the first magnet through the port to the compartment 6. FIG. 19e shows how the attraction between the magnets transports the second magnet to locate the collected magnetic particles in the imaging plane of the detector 9, where the magnetic particles and any analyte and labels are analysed. The second magnet 2 may be released from the imaging plane by using the magnet location fixture 8 to move the magnet temporarily from the wall of the compartment 6.

FIGS. 20a-d show the collection of magnetic particles from the sample with a second magnet in the cap of a sample container. FIG. 20a shows how the sample 1 is mixed with magnetic particles and labels with affinity for the analyte 1 in a container with a second magnet 2 in a housing 3 in the cap 4 of the sample container. FIG. 20b shows how the magnetic particles and any bound analyte and label are collected by the second magnet 2 by inverting the sample container such that the sample 1 contacts the second magnet 2 through a port 5. The housing 3 and port 5 provide for the magnetic particles to be collected through the port 5 predominantly on a particular pole of the second magnet, which pole has the same magnetic polarity as the pole of the magnet 8 presented to the interior of the compartment 7, such that the magnetic pole in the housing around the second magnet is attracted to the opposite pole of the magnet 8 presented to the interior of the compartment 7. FIG. 20c shows how the second magnet may be transferred to a further medium to label the analyte bound to the magnetic particles collected by the magnet. FIG. 20d shows how the second magnet 2 is moved to the port of the compartment 7 attached to a device fitted with a camera, such as a mobile phone or smartphone 6. FIG. 20d shows how the attraction between the first and second magnets transports the second magnet 2 to locate the magnetic particles collected on the pole of the second magnet 2 in the imaging plane of the detector 10, where the magnetic particles and any analyte and labels are analysed. The second magnet 2 may be released from the magnet 8 and imaging plane by using the magnet location insert 9 to move the magnet temporarily from the wall of the compartment 7.

FIGS. 21a-e show the collection of magnetic particles from the sample with a second magnet in a housing on a dipstick. FIG. 21a shows how the sample is mixed with magnetic particles and labels with affinity for the analyte 1 in a container. FIG. 21b shows a second magnet 2 housed at the end of dipstick with a plunger 6 connected through the handle 5 of the dipstick 4 to be in proximity to the second magnet 2. FIG. 21c shows how the sample 1 is contacted by the dipstick such that the second magnet is immersed in the sample and magnetic particles and any bound analyte and label are collected predominantly on a particular pole of the second magnet 2, which pole has the same magnetic polarity as the pole of the magnet 10 presented to the interior of the compartment 8, such that the magnetic pole in the housing around the second magnet is attracted to the opposite pole of the magnet 10 presented to the interior of the compartment 8. FIG. 21d shows how the dipstick is removed from the sample 1, optionally placed in media to label the analyte not shown, as FIG. 19c, when the second magnet 2 is inserted into the port in the compartment 8 until the stop 3 contacts the outer wall of the compartment 8. The plunger 6 is pressed to contact and release the second magnet 2 into the compartment, when attraction between the magnets transports the second magnet 2 to locate the magnetic particles collected on the pole of the second magnet 2 into the imaging plane of the detector 9, see FIG. 21e, where the magnetic particles and any labelled analyte are analysed. The second magnet 2 may be released from the magnet 10 and imaging plane by using the magnet location insert 11 to move the magnet temporarily from the wall of the compartment 8.

FIGS. 22a-e show the collection of magnetic particles from the sample on a second magnet in a housing in the seal of a plunger in a syringe. FIG. 22a shows the syringe with a port to the sample 1 provided with a plunger 5 reaching through a backplane 4 mounting a seal 3 housing a second magnet 2. FIG. 22b shows how the sample 6 is charged into the syringe and mixed with magnetic particles and labels with affinity for the analyte by pulling the plunger 5 of the syringe, which retracts the seal 3 mounted on the backplane 4 into the syringe barrel. FIG. 22c shows how the syringe is inverted such that the second magnet 2 contacts the sample. FIG. 22d shows the plunger 5 partially depressed such that the seal remains in a fixed position and the plunger 5 and the second magnet 2 break free of the seal 3 to become immersed in the sample. Magnetic particles and any bound analyte and label are collected predominantly on a particular pole of the second magnet 2, which pole has the same magnetic polarity as the pole of the magnet 11 presented to the interior of the compartment, such that the magnetic pole in the housing around the second magnet is attracted to the opposite pole of the magnet 11 presented to the interior of the compartment 9. FIG. 22e shows how the syringe is inserted into the port of the compartment and the plunger 5 pressed fully to release 8 the second magnet 2 into the compartment 9, when attraction between the magnets transports the second magnet 2 to locate the magnetic particles collected on the pole of the second magnet 2 in the imaging plane of the detector 10, where the magnetic particles and any labelled analyte are analysed. The second magnet 2 may be released from the magnet 11 and imaging plane by using the magnet location insert 12 to move the magnet temporarily from the wall of the compartment 9.

FIGS. 23a-c show the collection of magnetic particles from the sample on a second magnet in a housing in the seal of plunger contacting a toroidal magnet in the compartment. In FIG. 23a, a syringe is provided with a plunger 5 reaching through a backplane 4 mounting a seal 3 housing a second magnet 2. The sample 1 is charged into the syringe and mixed with magnetic particles and labels with affinity for the analyte by pulling the plunger 5 of the syringe, which retracts the seal 3 mounted on the backplane 4 into the syringe barrel. The second magnet 2 contacts the sample with the syringe inverted. FIG. 23b shows how the plunger 5 is partially depressed such that the seal 3 remains in a fixed position and the plunger 5 and the second magnet 2 break free of the seal 3 to become immersed in the sample. Magnetic particles and any bound analyte and label are collected predominantly on a particular pole of the second magnet 2, which pole has the same magnetic polarity as the pole of the magnet 6 presented to the interior of the compartment, such that the magnetic pole in the housing around the second magnet is attracted to the opposite pole of the magnet 6 presented to the interior of the compartment 7. FIG. 23c shows how the syringe is inserted into the port of the compartment and the plunger 5 pressed fully to release the second magnet 2 into the compartment 7, when attraction between the magnets transports the second magnet 2 to locate the magnetic particles collected on the pole of the second magnet 2 in the imaging plane of the detector 8, where the magnetic particles and any labelled analyte are analysed, through the hole in the toroidal magnet 6.

FIGS. 24a-d show the collection of magnetic particles from a sample using a magnetic swab. In FIG. 24a, the sample 2 is contacted directly with magnetic particles and labels with affinity for the analyte 1, for example, by using a spray or dropping device. FIG. 24b shows a second magnet 3 housed at the end of swab with a plunger 6 connected through the handle of the swab 5 to be in proximity to the second magnet 3, which contacts the magnetic particles 1 on the sample 2. The second magnet is immersed in the magnetic particles and contacts and swabs the sample. Any analyte and label bound to the magnetic particles are collected predominantly on a particular pole of the second magnet 3, which is the same pole on the magnet 10 presenting to the interior of the compartment 8. In FIG. 24c, the swab is removed from the sample 2, optionally placed in media to label the analyte not shown, as FIG. 19c when the second magnet 3 is inserted into the port in the compartment until the stop 4 contacts the outer wall of the compartment 8. The plunger 6 is pressed to contact and release the second magnet 3 into the compartment, when the magnet 10 transports the second magnet 3 to locate the magnetic particles collected on the pole of the second magnet 3 in the imaging plane of the detector 9, where the magnetic particles and any labelled analyte are analysed. The second magnet 3 may be released from the magnet 10 and imaging plane by using the magnet location insert 11 to move the magnet temporarily from the wall of the compartment 8.

FIGS. 25a-c show the detection of Clostridium difficile spores swabbed from surfaces. C. difficile spores ˜200 were introduced onto FIG. 25a metal, FIG. 25b plastic, and FIG. 25c wood surfaces. The surfaces were swabbed. The spores were collected on magnetic particles modified with antibody against C. difficile spores. The C. difficile spores were treated with a fluorescent staining reagent [Aojula et al. (2012) Method Reagent & Apparatus for Detecting a Chemical Chelator. Int. Pat. Appl. WO 2012/017194 A1]. Images were taken of the spores captured on magnetic beads and collected on a second magnet transported into the imaging plane of a mobile camera. The image shows fluorescent C. difficile spores 2 captured on magnetic particles collected on the surface of the reflective magnet. The boundary of the magnet is not visible. The approximate boundary is indicated by the dotted line 1 for clarity. The number of captured spores recovered from the contaminated surface can be enumerated.

FIGS. 26a-c show collection of magnetic particles in a well with an air/liquid interface providing a reflective surface. More specifically, FIG. 26a shows the well 3 attached to a housing 1 of a magnet 2. FIG. 26b shows the well filled with sample mixed with magnetic binding ligand 4 providing an air/liquid interface 5. FIG. 26c shows the well 3 detached from the magnet housing 1 and inserted into the compartment 7, such that the air/liquid interface 5 faces a toroidal magnet 6 in the compartment 7. The toroidal magnet attracts magnetic particles and any bound analyte and label through the liquid to the interface with air, where the particles are imaged through the hole in the toroidal magnet and anlaysed in the camera device 9.

It is to be appreciated that the embodiments of the invention described above have been given by way of example only and that modifications may be effected. Individual components shown in the drawings are not limited to use in their drawings and they may be used in other drawings and in all aspects of the invention.

1.-41. (canceled)

42. Apparatus for detecting an analyte using a fluid having magnetic particles with a binding ligand for binding the analyte, which apparatus comprises:
(i) a compartment;
(ii) a first magnet which is positioned outside of the compartment;
(iii) a second magnet with a reflective surface;
(iv) an inlet port for allowing the second magnet to enter the compartment and locate with a magnetic pole proximal to a magnetic pole of the first magnet; and
(v) the second magnet being for transporting on its reflective surface the magnetic particles with the binding agent and the analyte to an imaging plane where the particles are able to be illuminated for the detection of the analyte.

43. Apparatus according to claim 42 wherein the first magnet is an electromagnet.

44. Apparatus according to claim 42 and including imaging optics for the detection of the analyte.

45. Apparatus according to claim 44 in which the imaging optics include a telecommunications means.

46. Apparatus according to claim 42 and including illumination means.

47. Apparatus according to claim 46 in which the illumination means is a polarising means.

48. A method for detecting an analyte using a fluid having magnetic particles with a binding ligand for binding the analyte, which method comprises:
(i) providing the fluid;
(ii) providing a compartment with a first magnet;
(iii) contacting the fluid with a second magnet with a reflective surface locating the magnetic particles with a binding ligand for binding the analyte;
(iv) putting the second magnet in proximity to an inlet in the compartment;
(v) transporting the magnetic particles with the binding ligand into an imaging plane, using the first magnet to locate in the imaging plane; and
(vi) arranging the imaging plane detected to comprise light scattered by the magnetic particles with the binding ligand.

49. A method according to claim 48 and including providing primary imaging optics for the detection of the analyte.

50. A method according to claim 48 wherein the fluid is a liquid including a second labelled binding ligand with a different specific affinity for the analyte.

51. A method according to claim 50 wherein the magnetic binding ligand and the second binding ligand have different specific affinities.

52. A method according to claim 50 wherein the magnetic binding ligand and the second binding ligand have the same specific affinity.

53. A method according to claim 48 wherein the binding ligand is antibody, drug, peptide, or nucleic acid.

54. A method according to claim 48 and further comprising contacting the fluid with a plurality of magnetic binding ligands with specific affinity for different analytes in a sample, and contacting the sample with a plurality of different labelled binding ligands with specific affinity for different analytes.

55. A method according to claim 54 wherein the sample is food, beverage, bodily fluid or an environmental sample.

56. A method according to claim 48 wherein the analyte is a protein, a nucleic acid, a virus, a bacterial cell, a spore or a fungus cell.
